(19) United States
(12) Patent Application Publication
(10) Pub. No.: US 2024/0002955 A1
(43) Pub. Date: Jan. 4, 2024

(54) Methods for Estimating Microbial Density in Specimens by Measurement of Ribosomal RNA

(71) Applicants: MicrobeDX, Inc., Pacific Palisades, CA (US); The Regents of the University of California, Oakland, CA (US)

(72) Inventors: Bernard Churchill, Los Angeles, CA (US); Scott Adam Churchman, Santa Monica, CA (US); David Arnold Haake, Culver City, CA (US); Colin Wynn Halford, Los Angeles, CA (US); Roger Knauf, Cincinnati, OH (US); Gabriel Monti, Cypress, CA (US); Victoria Scott, Los Angeles, CA (US)

(21) Appl. No.: 18/369,022

(22) Filed: Sep. 15, 2023

Related U.S. Application Data
(63) Continuation of application No. 17/054,288, filed on Nov. 10, 2020, now abandoned.
(60) Provisional application No. 62/671,380, filed on May 14, 2018.

(51) Int. Cl. C12Q 1/689

(52) U.S. Cl. CPC C12Q 1/689

(57) ABSTRACT

A method of determining a bacterial density in a specimen may include the steps of: (a) conducting an RNA assay on the specimen to determine a microbial rRNA concentration, wherein the microbial rRNA concentration is defined as the number of rRNA molecules per volume of the specimen; and (b) converting the rRNA concentration to a bacterial density value.

### CROSS-REFERENCE TO RELATED APPLICATION

The present application is a continuation of U.S. patent application Ser. No. 17/054,288, filed on Nov. 10, 2020, and entitled “Methods for Estimating Microbial Density in Specimens by Measurement of Ribosomal RNA,” which is a national stage entry of PCT/US2019/032235 and also claims the benefit under 35 U.S.C. § 119(e) of U.S. Provisional Patent Application No. 62/671,380, filed May 14, 2018, and entitled “Methods for Estimating Microbial Density in Specimens by Measurement of Ribosomal RNA,” the contents of which are hereby incorporated by reference.

### FIELD OF THE INVENTION

The present invention relates to a method for estimating bacterial or microbial density. More specifically, the invention relates to a method for estimating bacterial density in a specimen, and particularly a method for estimating bacterial density in a specimen and/or a clinical specimen, using a ribosomal RNA-based signal.

### BACKGROUND

United States patent publication no. US2015/0104789 describes probes and methods for detecting antibiotic susceptibility of a specimen. The method comprises contacting the specimen with an oligonucleotide probe that specifically hybridizes with a target nucleic acid sequence region of ribosomal RNA. The target sequence is at the junction between a pre-ribosomal RNA tail and mature ribosomal RNA of 23S or 16S rRNA. Performing the method in the presence and absence of an antibiotic permits detection of antibiotic susceptibility.

United States patent publication no. US2011/0111987 describes a microfluidic system for processing a sample that includes a microfluidic CD in the form a rotatable disc, the disc containing a plurality of separate lysis chambers therein. A magnetic lysis blade and lysis beads are disposed in each of the lysis chambers and a plurality of stationary magnets are disposed adjacent to and separate from the microfluidic CD. The stationary magnets are configured to magnetically interact with each of the magnetic lysis blades upon rotation of the microfluidic CD. Each lysis chamber may have its own separate sample inlet port or, alternatively, the lysis chambers may be connected to one another with a single inlet port coupled to one of the lysis chambers. Downstream processing may include nucleic acid amplification using thermoelectric heating as well as detection using a nucleic acid microarray.

PCT patent publication no. WO2016/085632 describes methods and devices for rapid assessment of whether a microorganism present in a sample is susceptible or resistant to a treatment.

### SUMMARY

This summary is intended to introduce the reader to the more detailed description that follows and not to limit or define any claimed or as yet unclaimed invention. One or more inventions may reside in any combination or sub-combination of the elements or process steps disclosed in any part of this document including its claims and figures.

Some methods of quantifying bacterial ribosomal RNA (“rRNA”) are known generally in the art. For example, Gau et al. (2001),¹ Gau et al. (2005),² and Liao et al. (2007)³ all describe methods of quantifying bacterial rRNA concentration. Such methods of quantifying rRNA concentration include the following four steps: 1) Lysis to release rRNA; 2) Neutralization; 3) Hybridization of target rRNA with a capture probe and detector probe; and 4) Detection of capture probe-target rRNA-detector probe complexes. Determination of rRNA concentration may be based on a linear log-log correlation between the assay signal and rRNA analyte concentration. A synthetic target molecule at a known concentration may be included as a positive control for normalization of assay signal intensity, whereby the assay signal generated by a sample may be ¹ Gau J J, Lan E H, Dunn B, Ho C M, Woo J C. A MEMS based amperometric detector for E. coli bacteria using self-assembled monolayers. Biosens Bioelectron. 2001; 16(9-12):745-55. PubMed PMID: 11679252.² Gau V, Ma S C, Wang H, Tsukuda J, Kibler J, Haake D A. Electrochemical molecular analysis without nucleic acid amplification. Methods. 2005; 37(1):73-83. PubMed PMID: 16213156.³ Liao J C, Mastali M, Li Y, Gau V, Suchard M, Babbitt J T, Gornbein J, Landaw E M, McCabe E R, Churchill B M, Haake D A. Development of an advanced electrochemical DNA biosensor for bacterial pathogen detection. J Mol Diagn. 2007; 9:158-68.

compared with the positive control result to determine the number of target rRNA molecules per volume tested (concentration).

Methods of identifying bacteria in specimens, including in clinical specimens, are also known generally in the art. For example, Liao et al. (2006)⁴ describes performing an rRNA detection assay directly on urine specimens from patients with a urinary tract infection (“UTI”) to identify the bacteria in the specimen. However, in Liao et al., specimens were refrigerated overnight before testing and no attempt was made to relate rRNA signal intensity to bacterial density. ⁴Liao J C, Mastali M, Gau V, Suchard M A, Moller A K, Bruckner D A, Babbitt J T, Li Y, Gornbein J, Landaw E M, McCabe E R, Churchill B M, Haake D A. Use of electrochemical DNA biosensors for rapid molecular identification of uropathogens in clinical urine specimens. J Clin Microbiol. 2006; 44(2):561-70. PubMed PMID: 16455913.

It is generally known that rRNA copies per cell may vary widely between specimens. For example, rRNA copies per cell in cultivated specimens may vary from as high as approximately 100,000 copies per cell to as low as approximately 6000 copies per cell, depending on the growth phase and density of bacteria cultivated in the growth medium.

Because of this wide variation in the number of rRNA copies per cell, known techniques of rRNA quantification would not have been expected to enable accurate determination of microbial density in a given specimen because the growth phase of the microbe in the specimen was unknown and likely to be variable. For example, rRNA quantification would not have been expected to enable accurate determination of bacterial density in urine specimens because the growth phase of bacteria in urine specimens was unknown, and rRNA quantification would not have been expected to enable accurate determination of bacterial density in a blood culture specimen produced in an effort to diagnose sepsis in a time-sensitive, clinical environment because the growth phase of bacteria in blood culture specimen was unknown. Accordingly, there remains a need to develop a method that can repeatably relate the rRNA concentration of a target microbe in a specimen (and preferably a clinical specimen) to the actual microbial density of the microbe in the specimen. This relation is preferably sufficiently accurate to help make decisions in a diagnosis and/or treatment of a subject from which the specimen was obtained.

In accordance with one broad aspect of the teachings described herein, a quantification curve that accurately relates rRNA concentration in a clinical specimen to bacterial density is described. For example, the methods described herein have been used to demonstrate rRNA quantification actually provides a reliable estimate of microbial density in a specimen.

Quantification of bacterial density may be an industry standard for testing of urine specimens, blood cultures and other specimens for the presence of bacteria or other microbes. Quantification may also help facilitate phenotypic antimicrobial susceptibility test (“AST”) assays to determine the correct inoculation of a clinical specimen into growth medium. For example, over inoculation of growth medium with bacterial cells may prevent growth of the cells and/or may prevent determination of antibiotic susceptibility.

It is an object of the present invention to provide a novel method for estimating microbial, optionally bacterial, density in a specimen.

It is another object of the present invention to provide a novel method for estimating microbial, optionally bacterial density in a specimen using an rRNA-based signal.

It is another object of the present invention to provide a novel method for estimating microbial, optionally bacterial density in a specimen using the rRNA concentration of microbe/bacteria in the specimen.

It is another object of the present invention to provide a novel method for estimating microbial, optionally bacterial density in a specimen using a species-specific rRNA concentration of one or more microbe/bacterial species present in the specimen.

It is another object of the present invention to provide a novel method for estimating microbial, optionally bacterial density in a specimen, wherein the estimation of microbe/bacterial density may be possible when the rRNA copies per cell may vary across specimens.

It is another object of the present invention to provide a novel method for quantifying microbial, optionally bacterial density in a specimen, wherein the number of rRNA copies per cell in the specimen may not be known.

It is another object of the present invention to provide a novel method for estimating microbial, optionally bacterial density in a specimen, wherein the growth phase of the microbe/bacteria in the specimen may not be known.

It is another object of the present invention to provide a novel method for estimating microbial, optionally bacterial density in a specimen, wherein the estimation of microbial/bacterial density may be completed in less than four (4) hours of obtaining the specimen.

It is another object of the present invention to provide a novel method for estimating microbial, optionally bacterial density in a specimen, wherein the estimation of microbial/bacterial density may be completed in less than three (3) hours of obtaining the specimen.

It is another object of the present invention to provide a novel method for estimating microbial, optionally bacterial density in a specimen, wherein the estimation of bacterial density may be completed in less than two (2) hours of obtaining the specimen.

It is another object of the present invention to provide a novel method for estimating microbial, optionally bacterial density in a specimen, wherein the estimation of bacterial density may be completed in less than one (1) hour of obtaining the specimen.

It is another object of the present invention to provide a novel method for estimating microbial, optionally bacterial density in a specimen, wherein the estimation of bacterial density may be completed in less than 30 minutes of obtaining the specimen.

It is another object of the present invention to provide a novel method for estimating microbial, optionally bacterial density in a specimen, wherein the estimation of bacterial density may be completed in less than 15 minutes of obtaining the specimen.

It is an object of the present invention to provide a novel method for estimating microbial, optionally bacterial density in a clinical specimen.

It is another object of the present invention to provide a novel method for estimating microbial, optionally bacterial density in a clinical specimen using an rRNA-based signal.

It is another object of the present invention to provide a novel method for estimating microbial, optionally bacterial density in a clinical specimen using the rRNA concentration of bacteria in the clinical specimen.

It is another object of the present invention to provide a novel method for estimating microbial, optionally bacterial density in a clinical specimen using a species-specific rRNA concentration of one or more microbial or bacterial species present in the clinical specimen.

It is another object of the present invention to provide a novel method for estimating microbial, optionally bacterial density in a clinical specimen, wherein the estimation of microbial or bacterial density may be possible when the rRNA copies per cell may vary across clinical specimens.

It is another object of the present invention to provide a novel method for quantifying microbial, optionally bacterial density in a clinical specimen, wherein the number of rRNA copies per cell in the clinical specimen may not be known.

It is another object of the present invention to provide a novel method for estimating microbial, optionally bacterial density in a clinical specimen, wherein the growth phase of the microbe or bacteria in the clinical specimen may not be known.

It is another object of the present invention to provide a novel method for estimating microbial, optionally bacterial density in a clinical specimen, wherein the estimation of microbial or bacterial density in the clinical specimen may then be used for application in a clinical setting.

It is another object of the present invention to provide a novel method for estimating microbial, optionally bacterial density in a clinical specimen, wherein the estimation of microbial or bacterial density in the clinical specimen may be accurate enough for further clinical actions regardless of the growth phase of the microbe or bacteria.

It is another object of the present invention to provide a novel method for estimating microbial, optionally bacterial density in a clinical specimen, wherein the estimation of microbial or bacterial density in the clinical specimen may be accurate enough for further clinical actions regardless of the density of microbe or bacteria in the clinical specimen.

It is another object of the present invention to provide a novel method for estimating microbial, optionally bacterial density in a clinical specimen, wherein the estimation of microbial or bacterial density in the clinical specimen may be accurate enough for further clinical actions regardless of the growth phase or density of the microbe or bacteria in the clinical specimen.

It is another object of the present invention to provide a novel method for estimating microbial, optionally bacterial density in a clinical specimen, wherein the estimation of microbial or bacterial density in the clinical specimen may then be used to determine the likelihood of microbial or bacterial infection.

It is another object of the present invention to provide a novel method for estimating microbial, optionally bacterial density in a clinical specimen, wherein the estimation of microbial or bacterial density in the clinical specimen may then be used to optimize the clinical specimen for one or more further clinical actions.

It is another object of the present invention to provide a novel method for estimating microbial, optionally bacterial density in a clinical specimen, wherein the estimation of microbial or bacterial density in the clinical specimen may be then be used to optimize the clinical specimen for antimicrobial susceptibility testing.

It is another object of the present invention to provide a novel method for estimating microbial, optionally bacterial density in a clinical specimen, wherein the estimation of microbial or bacterial density may be completed in less than four (4) hours of obtaining the clinical specimen.

It is another object of the present invention to provide a novel method for estimating microbial, optionally bacterial density in a clinical specimen, wherein the estimation of microbial or bacterial density may be completed in less than three (3) hours of obtaining the clinical specimen.

It is another object of the present invention to provide a novel method for estimating microbial, optionally bacterial density in a clinical specimen, wherein the estimation of microbial or bacterial density may be completed in less than two (2) hours of obtaining the clinical specimen.

It is another object of the present invention to provide a novel method for estimating microbial, optionally bacterial density in a clinical specimen, wherein the estimation of microbial or bacterial density may be completed in less than one (1) hour of obtaining the clinical specimen.

It is another object of the present invention to provide a novel method for estimating microbial, optionally bacterial density in a clinical specimen, wherein the estimation of microbial or bacterial density may be completed in less than 30 minutes of obtaining the clinical specimen.

It is another object of the present invention to provide a novel method for estimating microbial, optionally bacterial density in a clinical specimen, wherein the estimation of microbial or bacterial density may be completed in less than 15 minutes of obtaining the clinical specimen.

Accordingly, an aspect of the present invention provides a method of determining a bacterial density in a specimen, the method comprising:
conducting a rRNA assay on the specimen to determine a bacterial rRNA concentration, wherein the bacterial rRNA concentration is defined as the number of rRNA molecules per volume of the specimen; and
converting the rRNA concentration to a bacterial density value.

In another of its aspects, the present invention provides a method of determining a relationship between bacterial rRNA concentration and bacterial density in a group of specimens, the method comprising:
a. conducting a rRNA assay to determine a bacterial rRNA concentration in one or more specimens of a group of specimens, wherein the bacterial rRNA concentration is defined as the number of rRNA molecules per volume of the specimen;
b. converting the rRNA concentration in each specimen in the group to a bacterial density value; and
c. correlating the bacterial rRNA concentrations from (a) with the bacterial densities from (b).

In another of its aspects, the present invention provides a method of determining if a subject has an infection, comprising
a. conducting an RNA assay on a clinical specimen to determine a bacterial rRNA concentration, wherein the bacterial rRNA concentration is defined as the number of rRNA molecules per volume of the specimen;
b. converting the rRNA concentration to a bacterial density value; and
c. determining a likelihood of infection by comparing the bacterial density value with a predetermined infection threshold value.

In another of its aspects, the present invention provides a method of preparing a clinical specimen to be subjected to a direct-from-specimen phenotypic antimicrobial susceptibility test, comprising determining a dilution factor for inoculation, the method comprising:
a. conducting a rRNA assay on the specimen to determine a bacterial rRNA concentration, wherein the bacterial rRNA concentration is defined as the number of rRNA molecules per volume of the specimen; and
b. converting the rRNA concentration to a bacterial density value
c. outputting the bacterial density value in a format that is useful for determining the dilution factor for a phenotypic antimicrobial susceptibility test.

In another of its aspects, the present invention provides a method of determining a dilution factor of a clinical specimen to use in a direct-from-specimen phenotypic antimicrobial susceptibility test, the method comprising:
a. conducting a rRNA assay on the clinical specimen to determine a bacterial rRNA concentration, wherein the bacterial rRNA concentration is defined as the number of rRNA molecules per volume of the specimen; and
b. converting the rRNA concentration to a bacterial density value; and.
c. comparing the bacterial density value to a target inoculation concentration for use in a phenotypic antimicrobial susceptibility test.

In another of its aspects, the present invention provides a method of determining a microbial density in a specimen, the method comprising:
a. conducting a rRNA assay on the specimen to determine a microbial rRNA concentration, wherein the microbial rRNA concentration is defined as the number of rRNA molecules per volume of the specimen; and
b. converting the rRNA concentration to a microbial density value.

These and other aspects will become apparent to those of skill in the art upon reviewing the present specification.

### BRIEF DESCRIPTION OF THE DRAWINGS

Embodiments of the present invention will be described with reference to the accompanying drawings, in which:

FIG. 1, in a flowchart, illustrates the steps involved in estimating bacterial density in a urine specimen using the rRNA concentration of bacteria in the specimen;

FIG. 2, in a graph, illustrates the correlation between rRNA concentration and density of E. coli in urine specimens from patients with urinary tract infection;

FIG. 3, in a graph, illustrates the correlation between rRNA copies per cell and density of E. coli in urine specimens from patients with urinary tract infection; and

FIG. 4, in a graph, illustrates the contrast between rRNA copies per cell and density of E. coli cultivated in growth medium vs. E. coli in urine specimens from patients with urinary tract infection.

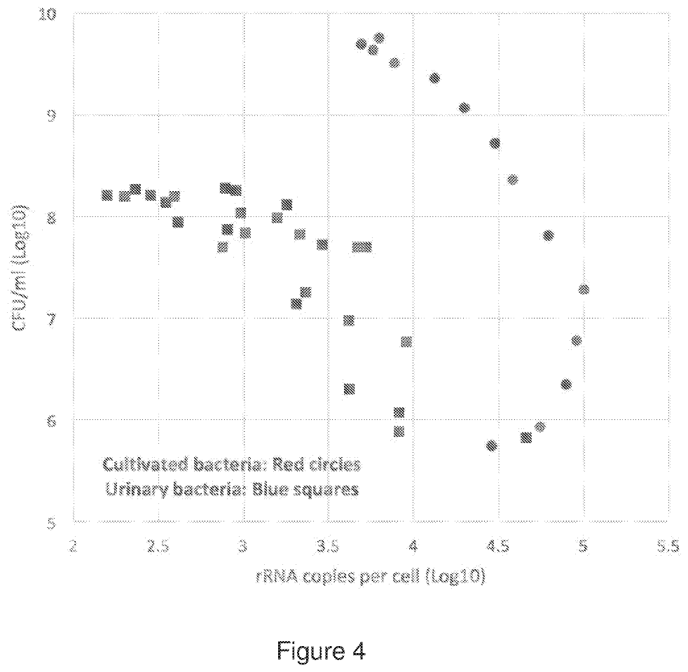

FIG. 5, in tabular form, illustrates the comparison between calculated CFU/ml concentrations values of clinical urine specimens with the actual CFU/ml from quantitative plate counting.

### DETAILED DESCRIPTION

The present invention relates to a method of determining a bacterial density in a specimen, the method comprising: (a) conducting a rRNA assay on the specimen to determine a bacterial rRNA concentration, wherein the bacterial rRNA concentration is defined as the number of rRNA molecules per volume of the specimen; and (b) converting the rRNA concentration to a bacterial density value. The method may further comprise comprising outputting the bacterial density value in a format that is useful for determining the dilution factor for a phenotypic antimicrobial susceptibility test.

Preferred embodiments of this method may include any one or a combination of any two or more of any of the following features:
a pre-determined translation function is used to convert the rRNA concentration to a bacterial density value;
steps (a) to (b) are conducted in sequence without an intervening step of culturing the specimen;
the specimen comprises at least one of a biological material and a culture of biological material obtained;
the rRNA assay produces an assay signal and wherein the bacterial rRNA concentration is based on a linear log-log correlation between the assay signal and an rRNA analyte concentration;
the bacterial rRNA concentration is determined by steps comprising (a) processing the bacterial rRNA in the specimen to obtain an rRNA signal; (b) taking the log of the rRNA signal to obtain an rRNA signal LOG; and (c) comparing the rRNA signal LOG with a positive control to determine the rRNA concentration of the specimen;
the rRNA signal is determined using an electrochemical sensor platform, an optical platform, or qRT-PCR.
the optical platform is an ELISA, magnetic beads, or capture probe array;
the rRNA is processed by steps comprising (a) lysing the specimen to release bacterial rRNA; (b) if necessary, neutralizing the released rRNA; (c) hybridizing the rRNA with capture and detector probes to form one or more capture probe-rRNA-detector probe complexes; and (d) detecting the resulting capture probe-rRNA-detector probe complexes;
the lysis of the bacteria comprises at least one of mechanical lysis, chemical lysis, and a combination of mechanical and chemical lysis;
the bacterial density value is determined from a pre-determined correlation between the bacterial rRNA concentration and the bacterial density;
the bacterial density value is determined by using a slope of a regression line from the pre-determined correlation between the bacterial rRNA concentration and bacterial density;
the slope of the regression line is a linear function;
the linear function has a formula y=mx+b, and wherein x in the formula is the bacterial rRNA concentration and y in the formula is the bacterial density value; the slope of the regression line is represented by the formula: y=1.79x+3.5;
determining a bacterial rRNA concentration of bacteria in the specimen and calculating a bacterial density value are completed in less than four (4) hours after obtaining a specimen, or in less than three (3) hours after obtaining a specimen, or in less than two (2) hours after obtaining a specimen, or in less than one (1) hour after obtaining a specimen, or in less than thirty (30) minutes after obtaining a specimen, or in less than fifteen (15) minutes after obtaining a specimen;
the specimen contains one bacterial species;
the specimen contains more than one bacterial species;
bacteria in the specimen have between about 1000 and about 100,000 rRNA copies each;
bacteria in the specimen have between about 5000 and about 45,000 rRNA copies each;
the bacterial density value is equal to the actual bacterial density in the specimen;
the bacterial density value is not equal to the actual bacterial density in the specimen;
the specimen is provided by or taken from a mammal;
the mammal is a human, dog, cat, murine, simian, farm animal, sport animal, or companion animal;
the specimen is a clinical specimen;
determining a bacterial rRNA concentration of bacteria in the specimen and calculating a bacterial density value are conducted directly on the clinical specimen;
the clinical specimen comprises a biological material; and/or
the biological material comprises at least one of urine, blood, blood culture, serum, plasma, saliva, tears, gastric fluids, digestive fluids, stool, mucus, sputum, sweat, earwax, oil, semen, vaginal fluid, glandular secretion, breast milk, synovial fluid, pleural fluid, lymph fluid, amniotic fluid, feces, cerebrospinal fluid, wounds, burns, tissue homogenates and an inoculum derived therefrom that is generated during conventional laboratory testing procedures.

In another of its aspects, the present invention relates to a method of determining a relationship between bacterial rRNA concentration and bacterial density in a group of specimens, the method comprising: (a) conducting an RNA assay to determine a bacterial rRNA concentration in one or more specimens of a group of specimens, wherein the bacterial rRNA concentration is defined as the number of rRNA molecules per volume of the specimen; (b) converting the rRNA concentration in each specimen in the group to a bacterial density value; and (c) correlating the bacterial rRNA concentrations from (a) with the bacterial densities from (b).

Preferred embodiments of this method may include any one or a combination of any two or more of any of the following features:
each specimen in the group contains one bacterial species;
the specimens contain more than one bacterial species;
the specimens are provided by or taken from mammals;
mammals are humans, dogs, cats, murines, simians, farm animals, sport animals, or companion animals;
each specimen in the group comprises a clinical specimens;
the clinical specimens are biological material;
the biological material comprises at least one of urine, blood, blood culture, serum, plasma, saliva, tears, gastric fluids, digestive fluids, stool, mucus, sputum, sweat, earwax, oil, semen, vaginal fluid, glandular secretion, breast milk, synovial fluid, pleural fluid, lymph fluid, amniotic fluid, feces, cerebrospinal fluid, wounds, burns, tissue homogenates and an inoculum derived therefrom that is generated during conventional laboratory testing procedures;
the bacterial rRNA concentration is based on a linear log-log correlation between an assay signal and an rRNA analyte concentration;
the bacterial rRNA concentration is determined for each specimen by steps comprising (a) processing the bacterial rRNA in the specimen to obtain an rRNA signal; (b) taking the log of the rRNA signal to obtain an rRNA signal LOG; and (c) comparing the rRNA signal LOG with a positive control to determine the rRNA concentration of the specimen.
the rRNA signal is determined using an electrochemical sensor platform, an optical platform, or a qRT-PCR;
the optical platform is an ELISA, magnetic beads, or capture probe array;
the rRNA is processed by steps comprising a) lysing the specimen to release bacterial rRNA; (b) neutralizing the released rRNA; (c) hybridizing the rRNA with capture and detector probes to form one or more capture probe-rRNA-detector probe complexes; and (d) detecting the resulting capture probe-rRNA-detector probe complexes;
the lysis of the bacteria is mechanical, chemical, or both mechanical and chemical;
the bacterial density of each specimen is determined by plate counts or microscopy;
the correlation between the bacterial rRNA concentrations and the bacterial densities is determined by plotting the log 10 of the bacterial rRNA concentration of each specimen against the log 10 of the bacterial density of each specimen;
the correlation between the bacterial rRNA concentrations and the bacterial densities has a linear relationship; and/or
the linear relationship is represented by the formula: y=1.79x+3.5, wherein x in the formula is the bacterial rRNA concentration and y in the formula is the bacterial density.

In another of its aspects, the present invention relates to a method of determining if a subject has an infection, comprising (a) conducting an RNA assay on a clinical specimen to determine a bacterial rRNA concentration, wherein the bacterial rRNA concentration is defined as the number of rRNA molecules per volume of the specimen; (b) converting the rRNA concentration to a bacterial density value; and (c) determining a likelihood of infection by comparing the bacterial density value with a predetermined infection threshold value. The method may further comprise outputting the bacterial density value in a format that is useful for determining the dilution factor for a phenotypic antimicrobial susceptibility test.

Preferred embodiments of this method may include any one or a combination of any two or more of any of the following features:
a pre-determined translation function is used to convert the rRNA concentration to a bacterial density value.
steps (a) to (b) are conducted in sequence without an intervening step of culturing the clinical specimen;
the specimen comprises at least one of a biological material and a culture of biological material;
the bacterial density value is determined from a known correlation between actual rRNA concentration and bacterial density;
the bacterial density value is determined by using a slope of a regression line from the known correlation between actual rRNA concentration and bacterial density;
the slope of the regression line is a linear function;
the slope of the regression line has a formula y=mx+b, and wherein x in the formula is the bacterial rRNA concentration and y in the formula is the bacterial density value;
the slope of the regression line is represented by the formula: y=1.79x+3.5;
an infection is likely if the bacterial density value is greater than or equal to the infection threshold value;
an infection is not likely if the bacterial density value is less than the infection threshold value;
the infection threshold value is 2 standard deviations above background;
the infection threshold value is 10,000 CFU/ml;
determining a bacterial rRNA concentration of bacteria in the specimen and calculating a bacterial density value in the clinical specimen are in less than four (4) hours after obtaining a clinical specimen, or in less than three (3) hours after obtaining a clinical specimen, or in less than two (2) hours after obtaining a clinical specimen, or in less than one (1) hour after obtaining a clinical specimen, or in less than thirty (30) minutes after obtaining a clinical specimen, or in less than fifteen (15) minutes after obtaining a clinical specimen;
the clinical specimen contains one bacterial species;
the clinical specimen contains more than one bacterial species;
bacteria in the specimen have between about 100 and about 100,000 rRNA copies each;
bacteria in the specimen have between about 5000 and about 45,000 rRNA copies each;
the specimen has a bacterial density and wherein the bacterial density value is equal to the actual bacterial density;
the specimen has a bacterial density and wherein the bacterial density value is not equal to the actual bacterial density;
the subject is a mammal;
the subject comprises at least one of a human, dog, cat, murine, simian, farm animal, sport animal, and a companion animal;
the clinical specimen comprises at least one of urine, blood, blood culture, serum, plasma, saliva, tears, gastric fluids, digestive fluids, stool, mucus, sputum, sweat, earwax, oil, semen, vaginal fluid, glandular secretion, breast milk, synovial fluid, pleural fluid, lymph fluid, amniotic fluid, feces, cerebrospinal fluid, wounds, burns, tissue homogenates and an inoculum derived therefrom that is generated during conventional laboratory testing procedures;
the bacterial rRNA concentration is based on a linear log-log correlation between an assay signal and an rRNA analyte concentration;
the bacterial rRNA concentration is determined by steps comprising (a) processing the bacterial rRNA in the specimen to obtain an rRNA signal; (b) taking the log of the rRNA signal to obtain an rRNA signal LOG; and (c) comparing the rRNA signal LOG with a positive control to determine the rRNA concentration of the specimen.
the rRNA signal is determined using an electrochemical sensor platform, an optical platform, or a qRT-PCR;
the optical platform is an ELISA, magnetic beads, or capture probe array;
the rRNA is processed by steps comprising (a) lysis to release rRNA of the bacteria in the specimen; (b) neutralization of lysate; (c) hybridization of target rRNA with capture and detector probes; and (d) detection of capture probe-rRNA-detector probe complexes; and/or
the lysis of the bacteria is mechanical, chemical, or both mechanical and chemical.

In another of its aspects, the present invention provides a method of determining a dilution factor of a clinical specimen to use in a direct-from-specimen phenotypic antimicrobial susceptibility test, the method comprising: (a) conducting a rRNA assay on the clinical specimen to determine a bacterial rRNA concentration, wherein the bacterial rRNA concentration is defined as the number of rRNA molecules per volume of the specimen; (b) converting the rRNA concentration to a bacterial density value; and (c) comparing the bacterial density value to a target inoculation concentration for use in a phenotypic antimicrobial susceptibility test.

Preferred embodiments of this method may include any one or a combination of any two or more of any of the following features:
the bacterial density value is greater than the target inoculation concentration;
the method further comprises the step of diluting the clinical specimen until the bacterial density value equal to or less than the target inoculation range;
the bacterial density value is equal to or less than the target inoculation concentration;
the method further comprises the step of preparing the inoculation without diluting the clinical specimen;
the target inoculation concentration is between about 1×105 CFU/ml to about 5×106 CFU/ml;
the target inoculation concentration is about 5×105 CFU/ml;
the clinical specimen is diluted with a growth medium;
the bacterial density value is determined from a known correlation between actual rRNA concentration and bacterial density;
the bacterial density value is determined by using a slope of a regression line from the known correlation between rRNA concentration and bacterial density;
the slope of the regression line is a linear function;
the slope of the regression line has a formula y=mx+b, and wherein x in the formula is the bacterial rRNA concentration in the specimen and y in the formula is the bacterial density value;
the slope of the regression line is represented by the formula: y=1.79x+3.5;
determining a bacterial rRNA concentration of bacteria in the specimen and calculating a bacterial density value in the clinical specimen are completed in less than four (4) hours after obtaining a clinical specimen, or in less than three (3) hours after obtaining a clinical specimen, or in less than two (2) hours after obtaining a clinical specimen, or in less than one (1) hour after obtaining a clinical specimen, or in less than thirty (30) minutes after obtaining a clinical specimen, or in less than fifteen (15) minutes after obtaining a clinical specimen;
the clinical specimen contains one bacterial species;
the clinical specimen contains more than one bacterial species;
bacteria in the clinical specimen have between about 1000 and about 100,000 rRNA copies each;
bacteria in the clinical specimen have between about 5000 and about 45,000 rRNA copies each;
the clinical specimen has a bacterial density and wherein the bacterial density value is equal to the actual bacterial density;
the specimen has an actual concentration of bacteria and wherein the bacterial density value is not equal to the actual concentration of bacteria in the specimen;
the clinical specimen is provided by or taken from a mammal;
the mammal is a human, dog, cat, murine, simian, farm animal, sport animal, or companion animal;
the clinical specimen comprises a biological material;
the biological material comprises at least one of urine, blood, serum, plasma, saliva, tears, gastric fluids, digestive fluids, stool, mucus, sputum, sweat, earwax, oil, semen, vaginal fluid, glandular secretion, breast milk, synovial fluid, pleural fluid, lymph fluid, amniotic fluid, feces, cerebrospinal fluid, wounds, burns, tissue homogenates and an inoculum derived therefrom that is generated during conventional laboratory testing procedures;
the bacterial rRNA concentration is based on a linear log-log correlation between an assay signal and an rRNA analyte concentration;
the bacterial rRNA concentration is determined by steps comprising: (a) processing the bacterial rRNA in the specimen to obtain an rRNA signal; (b) taking the log of the rRNA signal to obtain an rRNA signal LOG; and (c) comparing the rRNA signal LOG with a positive control to determine the bacterial rRNA concentration of the specimen;
the rRNA signal is determined using an electrochemical sensor platform, an optical platform, or qRT-PCR;
the optical platform is an ELISA, magnetic beads, or capture probe array;
the rRNA is processed by steps comprising (a) lysis to release rRNA of the bacteria in the specimen; (b) neutralization of lysate; (c) hybridization of target rRNA with capture and detector probes; and (d) detection of capture probe-rRNA-detector probe complexes; and/or
the lysis of the bacteria comprises at least one of mechanical lysis, chemical lysis and a combination of both mechanical and chemical lysis.

In another of its aspects, the present invention relates to a method of determining a microbial density in a specimen, the method comprising: (a) conducting an RNA assay on the specimen to determine a microbial rRNA concentration, wherein the microbial rRNA concentration is defined as the number of rRNA molecules per volume of the specimen; and (b) converting the rRNA concentration to a microbial density value. The method may further comprise outputting the microbial density value in a format that is useful for determining the dilution factor for a direct-from-specimen phenotypic antimicrobial susceptibility test.

Preferred embodiments of this method may include any one or a combination of any two or more of any of the following features:
a pre-determined translation function is used to convert the rRNA concentration to a microbial density value;
steps (a) to (b) are conducted in sequence without an intervening step of culturing the specimen;
the specimen comprises at least one of a biological material obtained from a subject prior to obtaining a specimen and a culture of biological material obtained from a subject that is produced prior to obtaining a specimen;
the rRNA assay produces an assay signal and wherein the microbial rRNA concentration is based on a linear log-log correlation between the assay signal and an rRNA analyte concentration;
the microbial rRNA concentration is determined by steps comprising: (a) processing the microbial rRNA in the specimen to obtain an rRNA signal; (b) taking the log of the rRNA signal to obtain an rRNA signal LOG; and (c) comparing the rRNA signal LOG with a positive control to determine the rRNA concentration of the specimen;
the rRNA signal is determined using an electrochemical sensor platform, an optical platform, or qRT-PCR;
the optical platform is an ELISA, magnetic beads, or capture probe array;
the rRNA is processed by steps comprising (a) lysing the specimen to release bacterial rRNA; (b) neutralizing the released rRNA; (c) hybridizing the rRNA with capture and detector probes to form one or more capture probe-rRNA-detector probe complexes; and (d) detecting the resulting capture probe-rRNA-detector probe complexes;
the lysis of the microbes comprises at least one of mechanical lysis, chemical lysis, and a combination of mechanical and chemical lysis;
the microbial density value is determined from a pre-determined correlation between the microbial rRNA concentration and the microbial density;
microbial density value is determined by using a slope of a regression line from the pre-determined correlation between the bacterial rRNA concentration and microbial density;
the slope of the regression line is a linear function;
the linear function has a formula y=mx+b, and wherein x in the formula is the microbial rRNA concentration and y in the formula is the microbial density value;
the slope of the regression line is represented by the formula: y=1.79x+3.5;
determining a microbial rRNA concentration of a microbe in the specimen and calculating a microbial density value in the specimen are completed in less than four (4) hours after obtaining a specimen, or in less than three (3) hours after obtaining a specimen, or in less than two (2) hours after obtaining a specimen, or in less than one (1) hour after obtaining a specimen, or in less than thirty (30) minutes after obtaining a specimen, or in less than fifteen (15) minutes after obtaining a specimen;
the specimen contains one microbial species;
the specimen contains more than one microbial species.
microbes in the specimen have between about 1000 and about 100,000 rRNA copies each;
microbes in the specimen have between about 5000 and about 45,000 rRNA copies each;
the microbial density value is equal to the actual microbial density in the specimen;
the microbial density value is not equal to the actual microbial density in the specimen;
the specimen is provided by or taken from a mammal;
the mammal is a human, dog, cat, murine, simian, farm animal, sport animal, or companion animal;
the specimen is a clinical specimen;
determining a microbial rRNA concentration of a microbe in the specimen and calculating a microbial density value in the specimen are conducted directly on the clinical specimen;
the clinical specimen comprises a biological material; and/or
the biological material comprises at least one of urine, blood, blood culture, serum, plasma, saliva, tears, gastric fluids, digestive fluids, stool, mucus, sputum, sweat, earwax, oil, semen, vaginal fluid, glandular secretion, breast milk, synovial fluid, pleural fluid, lymph fluid, amniotic fluid, feces, cerebrospinal fluid, wounds, burns, or tissue homogenates and an inoculum derived therefrom that is generated during conventional laboratory testing procedures.

Various apparatuses or processes will be described below to provide an example of an embodiment of each claimed invention. No embodiment described below limits any claimed invention and any claimed invention may cover processes or apparatuses that differ from those described below. The claimed inventions are not limited to apparatuses or processes having all of the features of any one apparatus or process described below or to features common to multiple or all of the apparatuses described below. It is possible that an apparatus or process described below is not an embodiment of any claimed invention. Any invention disclosed in an apparatus or process described below that is not claimed in this document may be the subject matter of another protective instrument, for example, a continuing patent application, and the applicants, inventors, or owners do not intend to abandon, disclaim, or dedicate to the public any such invention by its disclosure in this document.

The term “specimen” used herein refers to a material which is isolated from its natural environment, including but not limited to biological materials (see definition of “clinical specimen” below), food products, and fermented products.

The term “clinical specimen” used herein refers to samples of biological material, including but not limited to urine, blood, blood cultures (such as may be prepared when diagnosing sepsis), cultures of other biological material, serum, plasma, saliva, tears, gastric and/or digestive fluids, stool, mucus, sputum, sweat, earwax, oil, semen, vaginal fluid, glandular secretion, breast milk, synovial fluid, pleural fluid, lymph fluid, amniotic fluid, feces, cerebrospinal fluid, wounds, burns, tissue homogenates and/or an inoculum derived therefrom that is generated during conventional laboratory testing procedures. The clinical specimen may be collected and stored by any means, including in a sterile container.

A clinical specimen may be provided by or taken from any mammal, including but not limited to humans, dogs, cats, murines, simians, farm animals, sport animals, and companion animals.

The term “microbe” used herein refers to any species of microorganism, including but not limited to bacteria, fungi, and parasites.

The term “microbial density” used herein refers to the actual concentration of a given microbe in a specimen. Microbial density is expressed herein in colony forming units per milliliter (CFU/ml) but can be expressed by any another units, including but not limited to genomes per milliliter.

The term “microbial density value” used herein refers to an estimate or approximation of the microbial concentration in a specimen. The microbial density value may refer to a species-specific concentration of microbes or may refer to the concentration of more than one species/type of microbes. Microbial density value is expressed herein in colony forming units per milliliter (CFU/ml) but can be expressed by any another units, including but not limited to genomes per milliliter. As shown herein, the microbial density value may be equal to than the actual concentration of the microbial in a given specimen, or may be different.

The term “bacteria” used herein refers to any species of bacteria, including but not limited to Gram-negative and Gram-positive bacteria, anaerobic bacteria, and parasites.

The term “bacterial density” used herein refers to the actual concentration or quantity of bacteria in a specimen. Bacterial density is expressed herein in colony forming units per milliliter (CFU/ml) but can be expressed by any another units, including but not limited to genomes per milliliter.

The term “bacterial density value” used herein refers to an estimate or approximation of the bacterial concentration in a specimen. The bacterial density value may refer to a species-specific concentration of bacteria or may refer to the concentration of more than one species of bacteria. Bacterial density value is expressed herein in colony forming units per milliliter (CFU/ml) but can be expressed by any another units, including but not limited to genomes per milliliter. As shown herein, the bacterial density value may be equal to than the actual concentration of the bacteria in a given specimen, or may be different.

The term “rRNA” used herein refers to the ribosomal ribonucleic acid of bacteria present in a specimen.

The term “rRNA concentration” used herein refers to the number of rRNA molecules per volume tested. rRNA concentration is expressed herein in picomolar (pM) units but can be expressed by any another units.

The term “rRNA signal” used herein refers to the rRNA analyte concentration determined by the quantification of rRNA concentration in a specimen. An rRNA signal can be quantified by any known or unknown platform or method. Known platforms include but are not limited to electrochemical sensor platforms, optical platforms (e.g. ELISA, magnetic beads, capture probe arrays), and qRT-PCR.

The term “positive control” used herein refers to a known concentration of a target molecule that is included in an assay to produce a known and expected effect. Examples of target molecules that can be used as positive controls would be known to the person skilled in the art, and include synthetic oligonucleotides that have the same sequence as the target rRNA sequence.

The term “negative control” used herein refers to a known treatment that is included in an assay that is not expected to have any effect. Examples of treatments that can be used as negative controls would be known to the person skilled the art, and include specimens that do not contain rRNA, including RNase-treated samples.

The term “background” used herein refers to the result obtained from samples lacking rRNA, bacteria, or other microbes.

The term “infection threshold” used herein refers to the minimum microbial or bacterial density in a clinical specimen that indicates the presence of infection. A clinical specimen with a microbial or bacterial density above the “infection threshold” therefore may suggest the presence of infection. Microbial or bacterial densities below the cutoff may be considered negative for infection, possibly indicating such factors as contamination of the specimen during collection or outgrowth of contaminants during storage or transport. The infection threshold and how it is determined may differ for the type of specimen being analyzed, for the species of bacteria or microbe being analyzed, and/or for the infection being tested for. For example, when assessing for the presence of a urinary tract infection, a false negative rate of ≤5% may often be sufficient for tests for bacteriuria, which may be achieved by setting the infection threshold to 2 standard deviations above background.

The term “target inoculation concentration” used herein refers to the concentration of bacteria or microbe in a clinical specimen, or a range of concentrations of bacteria in a clinical specimen, that, when inoculated into growth medium, may provide accurate results on an antimicrobial susceptibility test (“AST”). For example, for a direct-from-specimen phenotypic AST of a specimen, an inoculation concentration may be between about 1×105 and about 5×106 CFU/ml, and preferably may be about 5×105 CFU/ml, and may provide an accurate/useful AST result, whereas inoculation concentrations more than 5×106 CFU/ml may reduce the accuracy. The target inoculation concentration may be used to determine what dilution factor, if any, is required to dilute a clinical specimen such that the bacterial density of the specimen may be optimized for an AST.

In some circumstances, time may be of the essence when detecting the presence of bacteria or other microbes in specimens. For example, such detection is often the first step in the diagnosis and/or treatment of infectious disease such as sepsis. A given clinical specimen, such as a direct bodily fluid sample or culture of blood or other bodily fluid sample, may be been obtained from a subject, whether it be a human or an animal, who may require further medical treatment based on the results of the analysis of the clinical specimen. For example, urine specimens are often obtained from subjects experiencing symptoms consistent with urinary tract infections. Similarly, blood samples are often obtained from subjects experiencing symptoms consistent with sepsis and blood cultures produced therefrom. Accurately determining the presence of bacteria, or combination of bacteria, and preferably a quantum of bacterial concentration, in such clinical specimens may help determine an appropriate course of treatment. For example, information regarding the bacterial density in a particular clinical specimen may be incorporated into the performance of an AST of significant bacterial isolates. The goals of such analyses are often to detect possible drug resistance in common pathogens and to assure susceptibility to drugs of choice for particular infections. This information may help clinicians prescribe effective antibiotics or other treatment regimes.

Conventional methods for determining the bacterial density in a sample often include at least one growth phase, in which a bacterial culture is prepared from the specimen. Such methods may be relatively accurate but are relatively slow, taking several hours, days, or weeks to provide useful results. In a clinical environment, such time frames may be undesirable and may be considered too long a time period to withhold/delay treatment for a subject. That is, while conventional techniques for determining bacterial density may tend to produce generally accurate results, they may be considered too slow to be of practical assistance. This time delay can sometimes lead to treatments being implemented, such as a particular antibiotic being prescribed, before the bacterial test results are obtained. This may lead to the unnecessary prescription of antibiotics and/or the prescription of a selected antibiotic that is less effective in treating a particular bacterium than other available antibiotics.

In addition to the time required to perform the analysis, conventional techniques often require a skilled technician to set-up and run the bacterial cultures, as well as to interpret the results. The analysis may also require specialized and/or costly equipment. As such equipment and skilled technicians can be relatively scarce resources, they are often located in centralized labs and/or hospital environments which are removed from common frontline care facilities, such as a physician's or veterinarian's office, walk-in clinics, and the like. This arrangement can further delay the processing and analysis of clinical specimens by several hours or days, as the specimens must be physically transported from the front line environment to a centralized testing location and may then wait in a testing queue or backlog of samples awaiting analysis. This time-delay may reduce the accuracy of the ensuing clinical specimen analysis due to such factors as growth or death of any bacteria that may be present in the specimen.

There remains a need for relatively faster specimen analysis methods, and a need to be able to perform at least some of the analysis in situ in a front line setting, such as in a physician's or veterinarian's office, instead of having to physically transport the specimens to a centralized location. Similarly, it would be advantageous to provide a method in which a clinically meaningful test result (i.e. information that can help inform treatment decisions) can be provided to a caregiver without requiring the individual skill and judgment of a skilled technician.

To help overcome at least some of these deficiencies in conventional methods of specimen analysis, the present inventors have developed a method in which it may be possible to estimate the bacterial density in a specimen in situ, in a front line setting, and in less time than conventional methods may allow for. In contrast to the established practices of quantifying bacterial density, the present inventors have discovered that rRNA quantification can provide a sufficiently reliable estimate of microbial density in a specimen so as to provide a meaningful measure of microbial density that may be useful for informing further actions (e.g. in clinical diagnosis and/or quantification of infections), and may be practically useful for other reporting, diagnosing, and/or therapeutic processes and methods. While experiments conducted to determine the bacterial density in urine samples are described in detail herein as one exemplary example, the methods and techniques described herein are applicable to a variety of different microbes.

The present inventors have determined that the number of rRNA copies per cell (z) may provide a link between the microbial rRNA concentration ([RNA]) and microbial density (CFU/ml) in a microbe-containing specimen, where it has been discovered that the number of rRNA copies per cell may be expressed as a Translation Function as follows:

[rRNA]=f(z)·cfu/ml

As indicated by this equation, the number of rRNA copies per cell (z) may be a linear function, which may be at least partially dependent on bacterial concentration. In some specimens, the microbe (e.g. bacterial) in the specimen have between about 1000 and about 100,000 rRNA copies each, or may have between about 5000 and about 45,000 rRNA copies each.

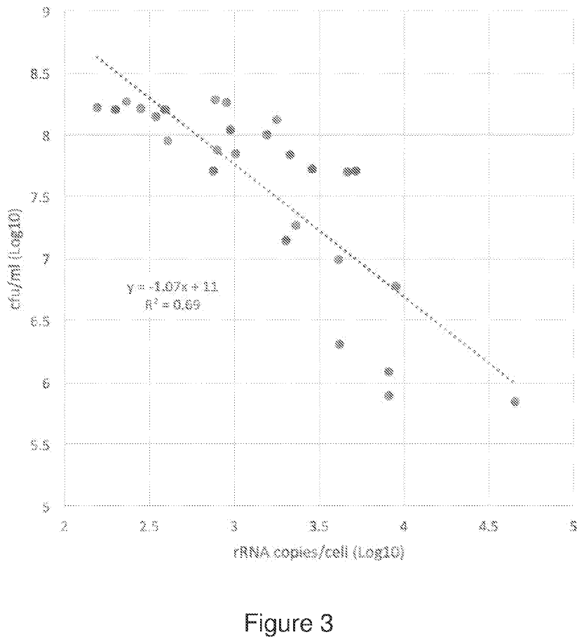

For example, it has been shown that the number of rRNA copies per cell (z) can provide a link between the microbial, or optionally bacterial rRNA concentration ([RNA]) and microbial, or optionally bacterial density (CFU/ml) in a microbial-containing specimen. FIG. 3 shows an equation that relates rRNA copies per cell to bacterial concentration in urine specimens.

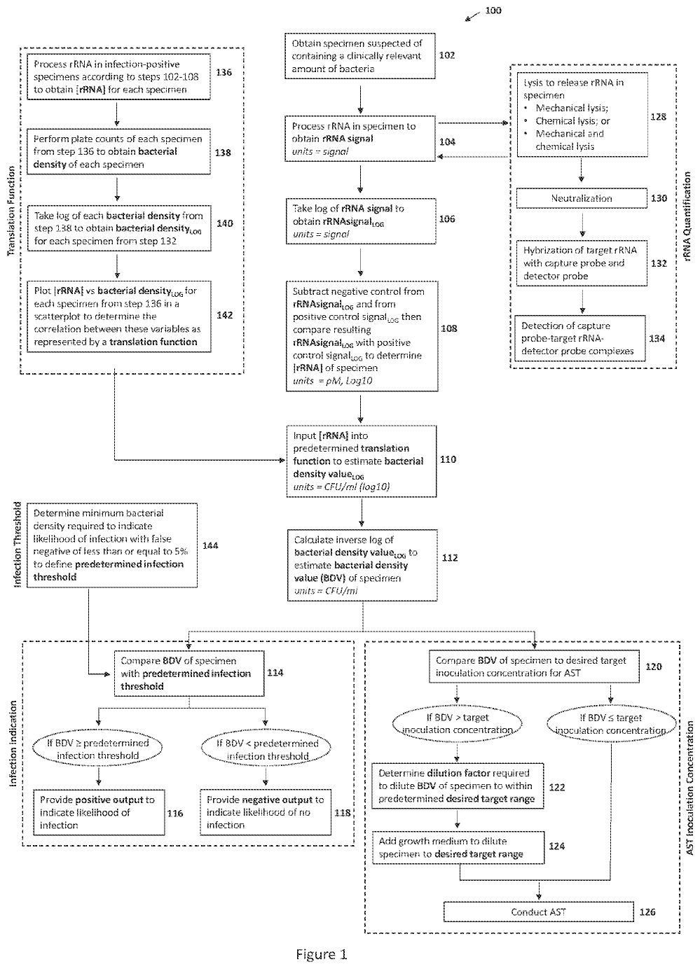

In accordance with one aspect of the teachings described herein, a method of quantifying bacterial density in specimen a urine specimen of a patient with a urinary tract infection (UTI) is described. FIG. 1 is a flowchart illustrating one embodiment of this method.

Referring to FIG. 1, one example of a method 100 of estimating the microbial density (in this case the bacterial density) in a clinical specimen includes a first step 102 of obtaining a clinical specimen. In most embodiments of the method, the clinical specimen is believed to contain at least one species of bacteria in a clinically relevant amount, and may be suspected of containing two or more species of bacteria in a clinically relevant amount. In the illustrated example, the clinical specimen is a urine specimen obtained from a patient that is complaining of symptoms consistent with a urinary tract infection and the specimen is suspected of containing at least a clinically relevant amount of E. coli.

Once the specimen suspected of containing a clinically relevant amount of bacteria is obtained, in a second step 104, the rRNA of the bacteria in the specimen is processed to obtain an rRNA signal. At least one positive control and at least one negative control are included in step 104.

In some embodiments of the invention, the time it takes from when a clinical specimen is obtained (i.e. step 102) to when the rRNA of at least one bacterial species in the clinical specimen has been processed (i.e. step 104) is less than four (4) hours. In some preferred embodiments, the time it takes from when a clinical specimen is obtained (i.e. step 102) to when the rRNA of at least one bacterial species in the clinical specimen has been processed (i.e. step 104) is less than 3 hours; less than 2 hours; less than 1 hour; less than 30 minutes; or less than 15 minutes.

The rRNA signal obtained from step 104 may then be used to determine the rRNA concentration of the bacteria in the specimen, preferably automatically when using a suitable system (i.e. without requiring intervention from a skilled technician). A determination of rRNA concentration may be based on a linear log-log correlation between the assay signal and the concentration of the rRNA analyte. Therefore, in a next step 106, the log of the rRNA signal from step 104 may be calculated to give the rRNA signal LOG.

In a next step 108, the log of the negative control signal from step 104 is subtracted from both the rRNA signal LOG from step 106 and the log of the positive control signal from step 104. The resulting rRNA signal LOG is then compared with the resulting positive control signal LOG to normalize the signal intensity of the rRNA signal LOG and determine the rRNA concentration of bacteria in the clinical specimen (units=pM, Log 10).

In a next step 110, the rRNA concentration from step 108 may be inputted into a predetermined translation function to estimate the bacterial density value LOG in the clinical sample (units=CFU/ml, Log 10).

In a next step 112, the inverse log of the bacterial density value LOG from step 110 may be calculated to estimate the bacterial density value of the clinical specimen (units=CFU/ml).

### RNA Quantification (Step 104)

Determining the concentration of rRNA may be done using any suitable method, including those described herein. One example of a suitable method may include the steps of: 1) Lysis to release rRNA 128; 2) Neutralization of the lysate 130; 3) Hybridization of target rRNA with a capture probe and detector probe 132; and 4) Detection of capture probe-target rRNA-detector probe complexes 134.

Optionally, the method of determining the concentration of the rRNA may be performed at least partially, and preferably completely, automatically using a suitable apparatus.

In the illustrated example, a MagPix (Luminex) magnetic bead assay is used to measure the E. coli rRNA concentration in fresh urine specimens from a patient with UTI.

### Lysis (Step 128)

Optionally, the lysing step 128 may include at least one of chemical lysing, mechanical lysing, and/or a combination thereof. In a preferred embodiment, lysis 128 may include both chemical and mechanical lysing operations. In a more preferred embodiment, the chemical and mechanical lysing operations may be performed simultaneously. Alternatively, the chemical and mechanical lysing operations may be performed at different times. One example of a suitable lysing method and apparatus is described in the U.S. provisional patent No. 62/541,418, which is incorporated herein by reference.

### Neutralization (Step 130)

The goal of the neutralization step is to get the lysate to a pH between about 6 and about, preferably about between 6.5 and about 7.5, most preferably, about 7. The neutralization step 130 can be performed using any known or unknown method.

In the illustrated example, samples are lysed with one-half sample volume of 1M NaOH. This lysate is then neutralized with an equal volume (1.5× sample volume) of 1M sodium-potassium phosphate buffer, pH 6.4.

### Hybridization (Step 132)

Preferably, a species-specific signal can be provided for each type of target bacteria that is expected to be present in the clinical specimen. By using a species-specific signal, the signal of rRNA from different types of bacteria in mixed specimens may be individually observed/counted and/or only signals from the desired, targeted bacteria may be counted. This may help facilitate the quantification of two or more different target bacteria within a common clinical specimen, and may allow the concentrations of two or more target bacterial rRNA concentrations to be measured generally simultaneously.

This may be advantageous when analyzing certain types of clinical specimens, such as urine specimens and/or blood culture specimens, which may tend to include a variety of different bacteria in generally unknown quantities at the beginning of the analysis process. By using species-specific signal probes, the methods described herein could be used to independently determine a quantity of rRNA from two or more specific bacterial species in the clinical specimen, input those values into respective, pre-determined transfer functions and calculate respective rRNA concentration values for each bacterial species. These results can then be used to provide outputs and/or as inputs in other method steps on a species-specific basis. For example, the methods may indicate a bacterial density value for E. coli that is above an E. coli pre-determined treatment threshold, while a bacterial density value for K. pneumoniae that is below its respective pre-determined treatment threshold. This may be used to initiate further treatment or diagnoses methods regarding E. coli, while not initiating analogous steps for K. pneumoniae. Alternatively, if both bacterial density values are above their respective pre-determined treatment thresholds, a different, suitable treatment protocol may be selected or followed.

### Detection (Step 134)

A variety of platforms can be used for detection 134, including but not limited to excitation and imaging of fluorescent-tagged detector probes, bioluminescence using luciferase-type enzymes, and amperometric current using an electrochemical sensor. In the illustrated example, fluorescent-tagged detector probes are used for detection.

During detection 134, at least one positive control and at least one negative control are included. In the illustrated example, a synthetic oligonucleotide with the same sequence as the target rRNA is included as a positive control and a sample without rRNA or bacteria is included as a negative control.

### Translation Function

The translation function used in step 110 is preferably selected from amongst one or more pre-determined translation functions. Suitable translation functions may be determined using any suitable technique, including those described herein. Optionally, more than one translation function may be determined and may be stored or otherwise recorded in a translation function table. For example, different translation functions may be developed for different species of bacteria that may be expected to be present in an incoming clinical specimen. That is, one translation function may be used to correlate the rRNA concentration and CFU/ml of E. coli in a given specimen, while a different translation function may be used to correlate the concentration of rRNA and CFU/ml of K. pneumoniae. Some translation functions may be better suited for use with a given type of bacteria.

Each translation function may take as an input a value that is based on the species-specific rRNA concentration in the specimen. For example, a translation function derived for E. coli may take as its input a value corresponding to the rRNA concentration of E. coli in the specimen, whereas a translation function for K. pneumoniae may take as its input a value corresponding to the rRNA concentration of K. pneumoniae in the specimen.

If more than one translation function has been determined, the methods and/or systems described herein may include the steps of selecting one translation function, from the two or more translation functions available, as being most appropriate for use with a given clinical specimen. The selection of a given translation function may be based on a variety of factors, including user inputs/selections, the expected types of bacteria, the type of specimen, ambient temperature, and sample storage time.

In a preferred embodiment, a translation function is derived from a microbe (e.g. bacterial) species-specific standard curve. To derive a microbe species-specific standard curve, rRNA concentrations of a specific microbe may be measured in a group of clinical specimens of the same type (e.g. a group of urine specimens). Species-specific microbe densities may then be determined on the same specimens using any known method. This relationship may then be plotted on a graph, with rRNA concentration (pM, Log 10) on one axis and CFU/ml (Log 10) on the other axis to determine the correlation between rRNA concentration and microbial density. The resulting relationship between these two variables may define a translation function.

The number of specimens required to derive a microbial species-specific standard curve may depend on such factors as the type of specimen and the species of bacteria being analyzed. The number of specimens required to accurately define a relationship between rRNA concentration and bacterial density may be determined using known statistical methods.

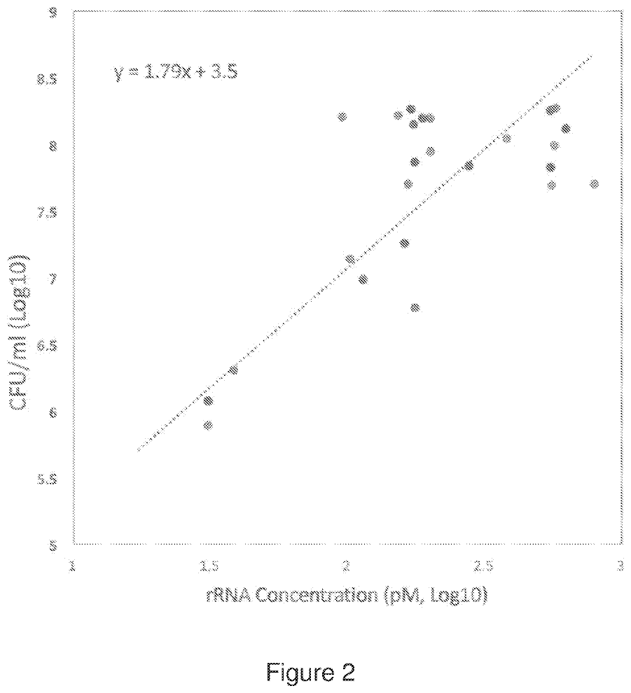

In the illustrated example, in a first step 136, a MagPix (Luminex) magnetic bead assay is used to measure E. coli rRNA concentrations in fresh urine specimens from 25 patients with UTI, as according to steps 102-108. In a next step 138, the bacterial density of E. coli in each specimen is determined with plate counts. In a next step 140, the log of each bacterial density from step 138 is calculated for each specimen to obtain the bacterial density LOG, which, in a next step 142, is plotted on a scatterplot against the rRNA concentration from step 136. From this scatterplot, the correlation between rRNA concentration and bacterial density is determined. FIG. 2 illustrates the correlation between E. coli rRNA concentration and density of E. coli for urine specimens from 25 patients with E. coli urinary tract infection.

In the illustrated example, the slope of the resulting regression line may be used as the translation function to estimate the E. coli bacterial density value (CFU/ml) in a urine specimen. More specifically, the linear equation of the resulting regression line, as represented by the general formula y=mx+b, may be used to estimate the bacterial density value (CFU/ml) of E. coli in a clinical specimen, wherein x is the rRNA concentration of E. coli in a clinical specimen (pM, Log 10) and y is the bacterial density value of E. coli in the clinical specimen (CFU/ml, Log 10).

In the illustrated example, the linear equation of the resulting regression line, and therefore the translation function, is y=1.79x+3.5, as seen in FIG. 2. Therefore, in the illustrated example, the translation function for E. coli was empirically determined to be y=1.79x+3.5, where y is CFU/ml (log 10) and x is the rRNA concentration (pM, Log 10) value for the tested clinical specimen. The bacterial density value in units of CFU/ml can then be obtained by taking the inverse log of y. In other words, the bacterial density value for E. coli can be described as:

bacterial density value=antilog(1.79x+3.5)

While in this example the x coefficient is presented with three significant digits, other examples of the translation function may have only a single decimal point or may be otherwise rounded while still providing a sufficiently accurate output for the bacterial density value on which to base clinical decisions.

### Microbial or Bacterial Density Value

Optionally, the microbial or bacterial density value (from step 112) can be provided to a user, for example via any suitable type of user display apparatus, such as a screen, print-out, email, text message, graphic, or the like. This information may then be used for any suitable purpose, including, for example, reporting and/or regulatory compliance.

In some embodiments, the microbial, or optionally bacterial density value may be used as an input or otherwise implicated in other sorts of methods. For example, in one embodiment, the microbial density value may be used to determine the likelihood of infection. In another embodiment, the microbial density value may be used as one of the inputs in a method or process that is to be performed on the clinical specimen. In another embodiment, the microbial density value may be used as a predictor of wound healing and/or acceptance of grafts.

### Screening for Infection

Estimation of microbial, or optionally bacterial density may be useful in testing clinical specimens for the presence of bacteria above a certain predetermined cutoff or threshold. Microbial densities above the cutoff may be considered positive and indicate the presence of infection; microbial densities below the cutoff may be considered negative and may indicate such factors as contamination of the specimen during collection or outgrowth of contaminants during storage or transport.

In the illustrated example in FIG. 1, at step 144, a false negative rate of ≤5% is determined to be sufficient to assess the likelihood of infection in a clinical specimen. This means that the cutoff for the assessment of infection is set to 2 standard deviations above background, meaning that if the bacterial density value of a specimen is greater than or equal to 2 standard deviations above background, there is a likelihood of infection. Conversely, if the bacterial density value of a specimen is less than 2 standard deviations above background, there is not a likelihood of infection.

In the illustrated example in FIG. 1, the likelihood of infection in a clinical specimen is assessed in steps 114-118. As a first step 114, the bacterial density value of E. coli in a urine specimen (from step 112) is compared with the predetermined infection threshold of 2 standard deviations above background (from step 144). If the bacterial density value from step 112 is greater than or equal to the infection threshold (i.e. ≥2 standard deviations above background), a positive output indicating the likelihood of infection is produced, as seen at step 116. Alternatively, if the bacterial density value from step 112 is less than the infection threshold (i.e. <2 standard deviations above background), a negative output indicating that infection is not likely is produced, as seen at step 118.

### AST Inoculation Concentration

Estimation of microbial, or optionally bacterial density may be useful in determining a dilution factor required for the inoculation of a clinical specimen into growth medium for a direct from specimen phenotypic antimicrobial susceptibility test (AST). Providing a bacterial density value that is within an acceptable resolution for clinical analysis may help determine an appropriate dosage of an inoculation agent to be used with a given clinical specimen to help provide a desired or target inoculation concentration in the clinical specimen. Utilizing the bacterial density value as a factor to help determine the dosage of the inoculation may help reduce the likelihood of over or under-diluting a given clinical specimen during further processing.

For example, in one embodiment, the target inoculation concentration of the AST may be 5×105 CFU/ml. Inoculation concentrations up to 5×106 CFU/ml may provide an accurate AST result, whereas inoculation concentrations greater than 5×106 CFU/ml may limit growth, thereby possibly reducing accuracy of AST results.

By using the techniques disclosed herein to make dilution calculations based on the bacterial density value, it may be possible to produce the same AST outcomes as would be expected by making dilution calculations based on the actual bacterial density—and/or based on currently accepted estimation techniques.

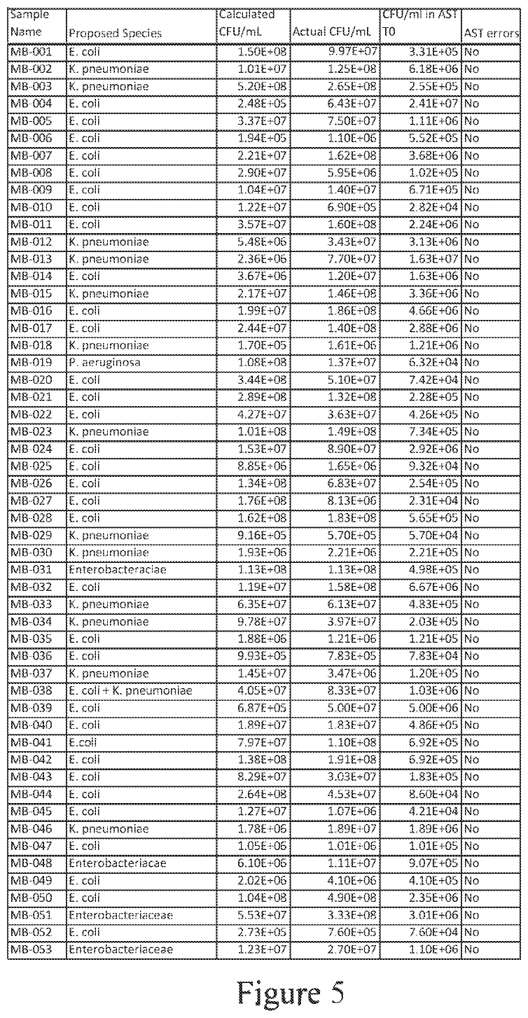

FIG. 5 shows the data generated from clinical urine specimens using the methodology described earlier. The calculated CFU/ml was determined by using the equation that relates the universal (EU) Luminex signal into a bacterial concentration. This can be compared to the actual CFU/ml, which was determined by diluting the cultures and counting the colonies formed on agar plates. The calculated concentration was used as a guide to dilute the urine specimen to ensure a starting AST concentration of 5×10\hat{ }5 CFU/ml. Even though there is some variation between the calculated and actual concentrations, the subsequently performed AST was not affected by the concentration difference and still produced accurate results.

In the illustrated example of FIG. 1, the determination of the AST inoculation concentration of the clinical specimen is set out in steps 120-126. As a first step, the bacterial density value from step 112 is compared to the predetermined desired target inoculation concentration for AST. If the bacterial density value from step 112 is greater than the desired target inoculation concentration, step 122 is engaged, in which the dilution factor required to dilute the bacterial density value of the specimen to within the desired target inoculation concentration range is determined. Based on the calculated dilution factor from step 122, growth medium is added to dilute the specimen to within the desired target range, as per step 124. The specimen can then be inoculated into growth medium for the AST, as per step 126.

On the other hand, if the bacterial density value from step 112 is less than or equal to the desired target inoculation concentration, the specimen may be inoculated into growth medium for the AST without dilution. In other words, steps 122-124 may be by-passed and the user would go immediately to step 126.

### Automation

Preferably, some or all of the steps in the methods can be automated using suitable equipment and do not require a skilled laboratory technician or the like to process the specimens and/or interpret the results. In some embodiments described herein, the inputs for the analysis method is a generally “fresh”, unmodified specimen obtained directly from a subject and the output of the method is an answer that is usable and/or understandable by a lay operator (i.e. not a skilled lab technician). For example, the output may be in the form of a number that represents the concentration of the target microbe or bacteria within the specimen.

What has been described above has been intended to be illustrative of the invention and non-limiting and it will be understood by the persons skilled in the art that other variants and modifications may be made without departing from the scope of the invention as defined by the claims appended hereto. The scope of the claims should not be limited by the preferred embodiments and examples, but should be given the broadest interpretation consistent with the description as a whole.

What is claimed is:

1. A method of determining a bacterial density value in a specimen, the method comprising:
(a) obtaining a specimen to be analyzed;
(b) conducting an RNA assay on the specimen including the steps of:
i) lysing the specimen to release bacterial rRNA using at least mechanical lysis;
ii) neutralizing the released rRNA;
iii) hybridizing the released rRNA with capture and detector probes to form one or more capture probe-rRNA-detector probe complexes; and
iv) detecting the capture probe-rRNA-detector probe complexes;
to determine a bacterial rRNA concentration in the specimen, wherein the bacterial rRNA concentration is defined as a number of rRNA molecules per volume of the specimen;
(c) converting the bacterial rRNA concentration to a bacterial density value; and
(d) displaying the bacterial density value to a user via a user display apparatus.

2. The method of claim 1, wherein a pre-determined translation function is used to convert the bacterial rRNA concentration to the bacterial density value.

3. The method of claim 1, the method comprising a further step of:
outputting the bacterial density value in a format that is useful for determining a dilution factor for a phenotypic antimicrobial susceptibility test.

4. The method described in claim 1, wherein the specimen comprises at least one of a biological material and a culture of biological material.

5. The method of claim 1, wherein the bacterial rRNA concentration is determined by steps comprising:
(a) processing the bacterial rRNA to obtain an rRNA signal, wherein the rRNA signal is determined using an electrochemical sensor platform, an optical platform, or a qRT-PCR, wherein the optical platform comprises at least one of an ELISA, magnetic beads, and a capture probe array;
(b) taking the log of the rRNA signal to obtain an rRNA signal_{LOG}; and
(c) comparing the rRNA signal_{LOG} with a positive control to determine the bacterial rRNA concentration of the specimen.

6. The method of claim 1, wherein a pre-determined correlation is used to convert the bacterial rRNA concentration to the bacterial density value.

7. The method of claim 1, wherein the specimen is provided by or taken from a mammal.

8. The method of claim 1, wherein the specimen is a clinical specimen and wherein steps a) and b) are conducted directly on the clinical specimen.

9. The method of claim 8, wherein the clinical specimen comprises a biological material, and the biological material comprises at least one of urine, blood, blood culture, serum, plasma, saliva, tears, gastric fluids, digestive fluids, stool, mucus, sputum, sweat, earwax, oil, semen, vaginal fluid, glandular secretion, breast milk, synovial fluid, pleural fluid, lymph fluid, amniotic fluid, feces, cerebrospinal fluid, wounds, burns, tissue homogenates and an inoculum derived therefrom that is generated during conventional laboratory testing procedures.

10. A method of determining if a subject has an infection, comprising
(a) conducting a rRNA assay on a clinical specimen from the subject including the steps of:
i) lysing the specimen to release bacterial rRNA using at least mechanical lysis;
ii) neutralizing the released rRNA;
iii) hybridizing the released rRNA with capture and detector probes to form one or more capture probe-rRNA-detector probe complexes; and
iv) detecting the capture probe-rRNA-detector probe complexes;
to determine a bacterial rRNA concentration, wherein the bacterial rRNA concentration is defined as a number of rRNA molecules per volume of the specimen;
(b) converting the bacterial rRNA concentration to the bacterial density value and displaying the bacterial density value to a user via a user display; and
(c) determining if the subject has the infection by comparing the bacterial density value with a predetermined infection threshold value of the subject related to the clinical specimen.

11. The method of claim 10, wherein a pre-determined translation function based on a known correlation between an actual rRNA concentration and a bacterial density is used to convert the bacterial rRNA concentration to the bacterial density value.

12. The method of claim 10, wherein the infection is likely if the bacterial density value is greater than or equal to the infection threshold value.

13. The method of claim 10, wherein an infection is not likely if the bacterial density value is less than the infection threshold value.

14. The method of claim 10, wherein the clinical specimen is obtained from a mammal.

15. The method of claim 10, wherein the clinical specimen comprises at least one of urine, blood, blood culture, serum, plasma, saliva, tears, gastric fluids, digestive fluids, stool, mucus, sputum, sweat, earwax, oil, semen, vaginal fluid, glandular secretion, breast milk, synovial fluid, pleural fluid, lymph fluid, amniotic fluid, feces, cerebrospinal fluid, wounds, burns, tissue homogenates and an inoculum derived therefrom that is generated during conventional laboratory testing procedures.

16. A method of determining a dilution factor of a clinical specimen to use in a direct-from-specimen phenotypic antimicrobial susceptibility test, the method comprising:
(a) conducting a rRNA assay on the clinical specimen to determine a bacterial rRNA concentration, wherein the bacterial rRNA concentration is defined as a number of rRNA molecules per volume of the specimen; and
(b) converting the bacterial rRNA concentration to a bacterial density value; and
(c) comparing the bacterial density value to a target inoculation concentration for use in a phenotypic antimicrobial susceptibility test, wherein
if the bacterial density value is greater than the target inoculation concentration then further including the step of diluting the clinical specimen so that the bacterial density value is equal to or less than the target inoculation concentration before conducting the phenotypic antimicrobial susceptibility test, or
if the bacterial density value is less than the target inoculation concentration then further including the step of conducting the phenotypic antimicrobial susceptibility test without diluting the clinical specimen.

17. The method of claim 16, wherein the clinical specimen is provided by or taken from a mammal.

18. The method of claim 16, wherein the clinical specimen comprises a biological material, and wherein the biological material comprises at least one of urine, blood, serum, plasma, saliva, tears, gastric fluids, digestive fluids, stool, mucus, sputum, sweat, earwax, oil, semen, vaginal fluid, glandular secretion, breast milk, synovial fluid, pleural fluid, lymph fluid, amniotic fluid, feces, cerebrospinal fluid, wounds, burns, tissue homogenates and an inoculum derived therefrom that is generated during conventional laboratory testing procedures.
